(19) United States
(12) Patent Application Publication
(10) Pub. No.: US 2024/0149059 A1
(43) Pub. Date: May 9, 2024

(54) ELECTRONIC REGIONAL ANESTHESIA AND PAIN MANAGEMENT SYSTEM AND METHOD

(71) Applicant: ALGIAMED LTD, Ebene (MU)

(72) Inventor: Corlius Birkill, Pretoria (ZA)

(21) Appl. No.: 18/386,731

(22) Filed: Nov. 3, 2023

Related U.S. Application Data
(60) Provisional application No. 63/422,205, filed on Nov. 3, 2022.

(51) Int. Cl. A61N 1/36; A61N 1/372

(52) U.S. Cl. CPC A61N 1/36017; A61N 1/36021; A61N 1/36031; A61N 1/36034; A61N 1/37247; A61N 1/0502

(57) ABSTRACT

A system and method for visualizing a position of a needle inside a patient's body for use in providing regional anesthesia and a system and method for providing electro anesthesia during surgery, post surgery and during rehabilitation. The method and system use EMG/AMG information regarding activity of a muscle associated with a target nerve to provide a visualization of a position of a needle or other device relative to the target nerve and to determine efficacy of the electro anesthesia. EMG/AMG information and feedback information such as EEG information may be used to provide automatic adjustment of a waveform provided for electro anesthesia to maintain a suitable pain level.

### CROSS-REFERENCE TO RELATED APPLICATION

The present application claims benefit of and priority to U.S. Provisional Patent Application Ser. No. 63/422,205 entitled ELECTRONIC REGIONAL ANESTHESIA AND PAIN MANAGEMENT SYSTEM AND METHOD filed Nov. 2, 2022, the entire content of which is hereby incorporated by reference herein.

### BACKGROUND

### Field of the Disclosure

The present disclosure relates to a system and method to provide visual guidance for a needle towards a targeted nerve utilizing a directional stimulating needle as a signal generator as well as EMG/AMG electrodes providing closed loop feedback as well as a system and method to provide electronic regional anesthesia during surgery, post-surgery and during rehabilitation, including a feedback system to provide suitable electrical signals to provide electronic regional anesthesia.

### Related Art

Regional anesthesia (RA) has increased its value in high-resource countries for the past 15 years thanks to its numerous benefits, ranging from improved analgesia to a decreased cost of care. Nowadays, RA is a fundamental procedure, popular worldwide in perioperative care due to its several advantages over general anesthesia. Among the reported benefits of RA, experts have found better patient outcomes and increased operating room efficiency.

Regional anesthesia has a number of benefits as opposed to general anesthesia as follows:
1. Fewer life-threatening perioperative airway and respiratory complications;
2. Decreased airway manipulation in high-risk patients—e.g. obstetrical, obese and COVID-19 patients;
3. Decreased blood loss during orthopedic surgery;
4. Lower incidence of blood clots after lower limb surgery;
5. Improved analgesia and decreased need for opioids;
6. Decreased stress-response to surgery;
7. Shortened length-of-stay in post-anesthesia care unit;
8. Resource sparing compared to general anesthesia with intubation;
9. Decreased mortality with regional anesthesia compared to general anesthesia with intubation in LRCs; and
10. Spinal anesthesia has benefits over ketamine anesthesia (commonly used in LRCs)
   These advantages are stimulating significant growth of this market segment compared to general anesthesia.

Some of the drawbacks associated with RA are rooted in the pharmaceuticals used. For example, there are risks involved in using local anesthetic drugs for short term, single shot regional anesthesia procedures due to their inherent toxicity. There are also issues related to using longer term drug induced regional anesthesia associated with the amount of toxicity introduced by the volume of drug required to maintain effective anesthesia/pain control over a prolonged period of time since this may exceed the toxicity threshold of the body.

A key consideration and difficulty associated with RA is the need to accurately identify and isolate the targeted nerve which presents a unique technical challenge. Ultrasound guidance may be used to aid in the delivering of analgesia and surgical anesthesia and adjustment of needle position by visualization while providing regional anesthesia. However, ultrasound-guided administration of RA increases the need for trained personnel who also require experience both in operating ultrasound machines generally and specialized training to identify anatomical structures using such machines. This requirement for specialized training represents an obstacle to using RA in low-resource countries or other area as well as limiting its use even in high resource countries.

Ultrasound guidance also provides limited improvement to nerve targeting. Indeed, results consistently show no difference in block success and/or pain scores when ultrasound guidance is added to the current standard procedure for RA. In fact, ultrasound guidance typically does not improve outcomes of RA in femoral nerve blocks, interscalene brachial plexus block, infraclavicular brachial plexus block, and popliteal sciatic nerve blocks. While ultrasound guidance adds a visual cue to the needle placement, it is highly patient and user-dependent; for example, parasacral sciatic, subgluteal sciatic and posterior lumbar plexus blockades are difficult to locate with ultrasound, especially on obese patients. In addition, the correct interpretation of needle position with ultrasound by expert anesthetists is estimated to be less than 80%. To this day, whether ultrasound-guidance improves anesthetists ability to perform peripheral nerve blocks remains controversial.

Another option is the use of percutaneous peripheral nerve stimulators that use electrical currents to locate a nerve prior to injection of anesthetics and to estimate the degree of neuromuscular block. Stimulation of peripheral nerves alone successfully locates nerves during blockade procedures using quantitative biomarkers, such as evoking motor response of associated muscles. Higher success rates and decreased patient discomfort are found when nerve identification is carried out using electrical stimulation compared to ultrasound-guided blockades. Another advantage of electrical stimulation over ultrasound guidance is the use of inexpensive equipment, when compared to ultrasound machines. However, there are limits to the use of electrical nerve stimulation guidance for blockage procedures related to possible vascular punctures, incorrect interpretation of the motor response, and incorrect selection of minimum threshold current which my give inconclusive or misleading results. Thus the use if peripheral nerve stimulation to guide injections during RA procedures offers its own technical problems.

Current nerve stimulators are used in a manual, iterative procedure where current of the stimulator is slowly decreased when an evoked motor response is observed. The needle is then maneuvered blindly until an increased evoked motor response is observed. The current is decreased again and the procedure repeated until a minimum current elicits the desired evoked response. At which time the proximity of the needle tip to the nerve is confirmed. This is an onerous approach and offers very similar accuracy to ultrasound guidance.

Accordingly, it would be beneficial to provide a method and system to provide visual guidance in identifying a targeted nerve and positioning a needle relative thereto to provide RA during surgery as well as postoperative pain treatment that avoids the use of local anesthetic drugs and the toxic nature thereof.

### SUMMARY

It is an object of the present disclosure to provide a method and system for visualizing needle position relative to a targeted nerve for use in providing regional electro-anesthesia during surgery as well as postoperative pain treatment.

A system for positioning a needle includes: a hollow needle configured to pierce a user's skin and including at least a first conductive surface adjacent to a point of the needle; a stimulator electrically connected to the first conductive surface and configured to provide an electrical signal to the first conductive surface; a feedback sensor configured to be secured to the user's skin and to provide stimulation information associated with activity of a muscle associated with a target nerve; a controller operably connected to the stimulator and the feedback sensor and configured to: provide stimulation parameters to the stimulator to control the electrical signal provided to the first conductive surface to provide a directional electrical field around the needle, receive stimulation information from the feedback sensor; and generate image information associated with a position of the needle in the user's body based on the stimulation parameters and stimulation information.

In embodiments, the system includes a display element operably connected to the controller and operable to receive the image information and provide an image of the needle in the user's body.

In embodiments, the controller includes: a processor; and memory operably connected to the processor and including processor executable code, that when executed by the processor: generates the stimulation parameters based at least on the stimulation information; and generates the image information based at least on the stimulation parameters and the stimulation information.

In embodiments, the image information includes distance information and direction information associated with the needle and the target nerve.

In embodiments, the processor executable code includes code that when executed by the processor, applies signal processing techniques to the stimulation information to generate the distance information and the direction information.

In embodiments, the processor executable code includes code that when executed by the processor, implements a machine learning algorithm to generate the distance information and the direction information based on the stimulation information and the stimulation parameters.

In embodiments, the stimulation parameters and stimulation information are provided to the display element and illustrated on the display element.

In embodiments, the display element is one of a liquid crystal display, cathode ray tube display, light emitting diode display and a plasma display.

In embodiments, the display element is a touch screen configured to enter information associated with the stimulation parameters.

In embodiments, an input element configured to receive the stimulation parameters.

In embodiments, the hollow needle is configured to provide a pharmaceutical anesthesia to the target nerve.

In embodiments, the system includes a catheter mounted in the needle with a distal end of the catheter extending beyond the hollow needle, the distal end including a conductive portion electrically connected to the stimulator, wherein the stimulator provides a pain relieving waveform to the conductive portion of the catheter for application to the target nerve.

In embodiments, the pain relieving waveform includes a low frequency square wave with a high frequency hi-polar exponentially decaying waveform superimposed thereon.

In embodiments, the high frequency hi-polar exponentially decaying waveform has a frequency of about 133 kHz.

In embodiments, the pain relieving waveform includes a radio frequency component between 50 kHz and 500 kHz of varying or nonvarying amplitude.

In embodiments, the stimulation information indicates effectiveness of the pain relieving waveform based on activity of the muscle associated with the target nerve.

In embodiments, the system includes a plurality of EEG electrodes configured for attachment to a user's head and configured to provide EEG information, indicating brain activity associated with pain of the user.

In embodiments, the stimulation parameters are set based on the FEG information.

In embodiments, the controller is operably connected to a user device associated with the user, wherein the user device provides control information to the controller to control the stimulator to provide the pain relieving waveform.

In embodiments, the user device is connected to the controller via a wired connection.

In embodiments, the user device is connected to the controller via a wireless connection.

In embodiments, at least one of the EEG information, brain stem response information, compound action potentials, nociceptive reflex information, EMG information and pupilometry information is provided to the display element and illustrated on the display element.

### BRIEF DESCRIPTION OF THE DRAWINGS

The above and related objects, features and advantages of the present disclosure will be more fully understood by reference to the following, detailed description of the preferred, albeit illustrative, embodiments of the present invention when taken in conjunction with the accompanying figures, wherein:

FIG. 1 illustrates an exemplary illustration of a system used to position a needle relative to a target nerve in accordance with an embodiment of the present disclosure;

FIG. 1A illustrates an exemplary block diagram of a system used to position a needle relative to a target nerve in accordance with an embodiment of the present disclosure;

FIG. 2 illustrates a directional needle used in the system of FIG. 1 in accordance with an embodiment of the present disclosure;

FIG. 2A illustrates a more detailed view of the end of the needle of FIG. 2 illustrating the conducting surfaces thereof in in accordance with an embodiment of the present disclosure;

FIG. 2C illustrates a stimulating catheter passed through the needle of FIGS. 2 and 2A;

FIG. 2D illustrates a more detailed view of an end of the stimulating catheter passed through the needle of FIGS. 2, 2A and 2C;

FIG. 3 illustrates a stimulating device suitable for use in the system of FIG. 1 in accordance with an embodiment of the present disclosure;

FIG. 4 illustrates an exemplary interface displayed on a display device suitable for use in the system of FIG. 1 to indicate a position of the needle relative to a target nerve in accordance with an embodiment of the present disclosure;

FIG. 5 illustrates an exemplary system for providing electro anesthesia suitable for use during surgery, post-surgery and in a rehabilitation environment in accordance with an embodiment of the present disclosure;

FIG. 6 illustrates a more detailed view of an EEG system suitable for use in the system of FIG. 5 in accordance with an embodiment of the present disclosure; and

FIG. 7 illustrates an exemplary illustration of an interface displayed on a display device indicating stimulation parameters, stimulation information and EEG information that may be used in the electro anesthesia system of FIG. 5.

### DETAILED DESCRIPTION OF THE EXEMPLARY EMBODIMENTS

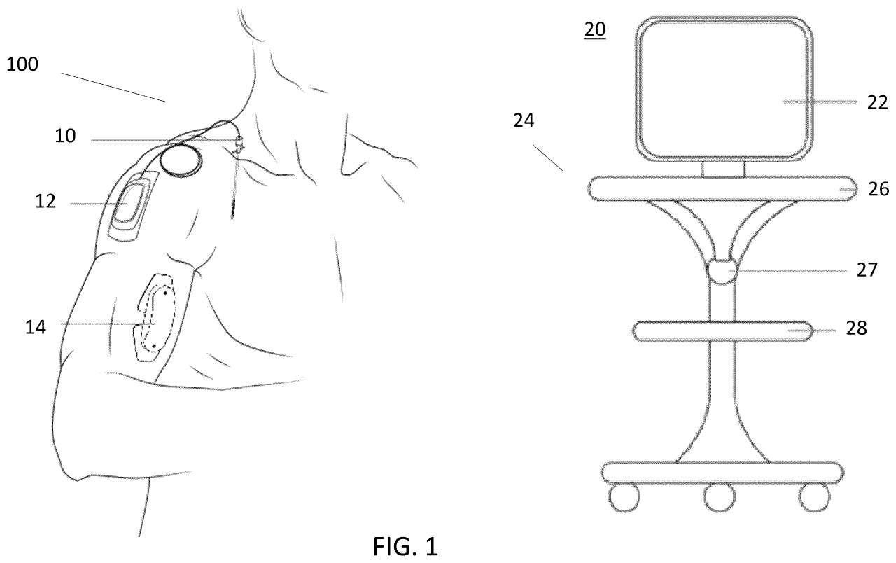

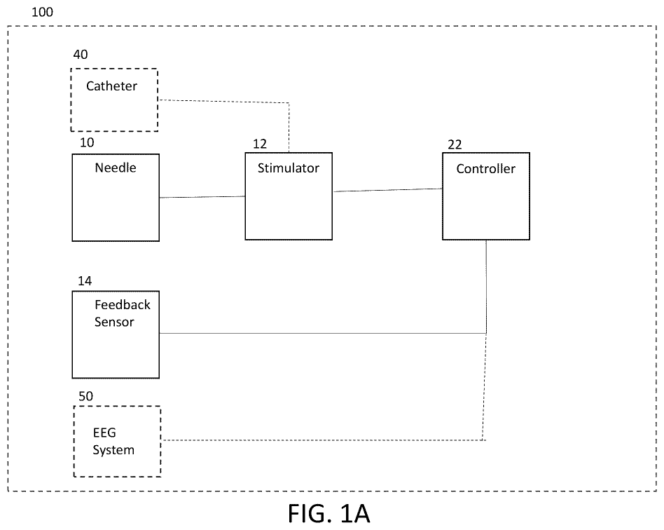

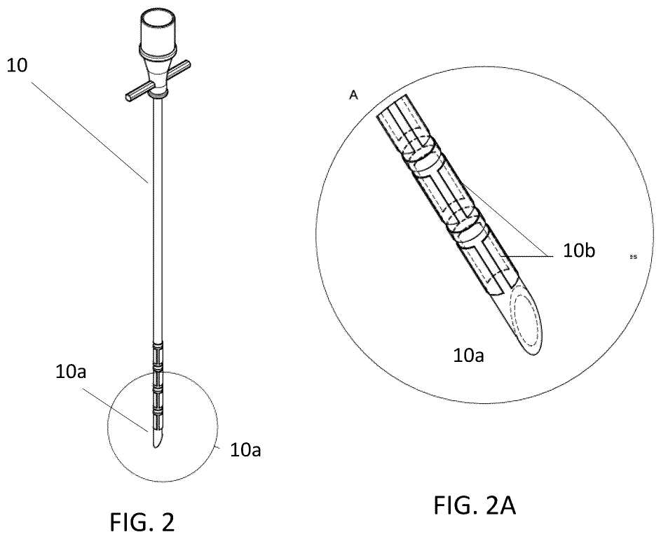

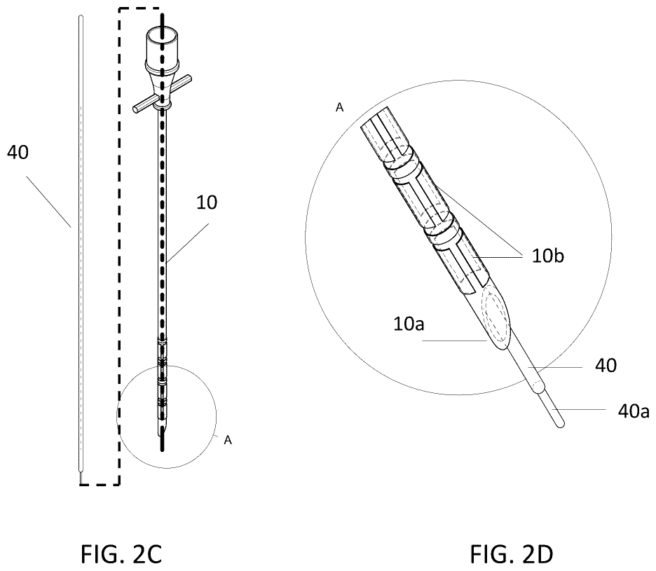

In embodiments, a visually aided, automated nerve stimulating nerve location system 100 is provided. In embodiments, the system 100 (see FIG. 1) uses electromyography (EMG) technology to objectively measure the evoked motor response of a muscle associated with a target nerve and a needle 10 with a plurality of conductive surfaces 10b (see FIG. 2A) which are offset in a way to allow for triangulation to provide a visual representation of the needle in the user's body. In embodiments, as noted below, acceleromyography (AMG) technology may also be used. In embodiments, the triangulation information may be utilised to calculate and generate image information to provide a visual representation of the distance between the needle tip 10a and the target nerve which may be shown to a user on display 22, for example.

In embodiments, the system 100 includes a needle 10 as illustrated in FIG. 1, for example. In embodiments, the distal end, or point 10a (see FIGS. 2-2A) of the needle 10 is configured to be inserted into the body of the user for positioning near the target nerve. In embodiments, the distal end 10a of the needle 10 includes a plurality of conducting surfaces 10b shown in more detail in FIG. 2A. In embodiments, the conducting surfaces 10b are offset from each other by 120 degrees to provide intermittent 360 degrees conducting bands. As can be seen in FIG. 2A, for example, a space may be provided between adjacent conducting surfaces 10b. In embodiments, any plurality of evenly spaced conducting surfaces 10b may be provided on the distal end 10a of the needle 10. In embodiments, the conducting surfaces 10b may be arranged in various patterns or positions.

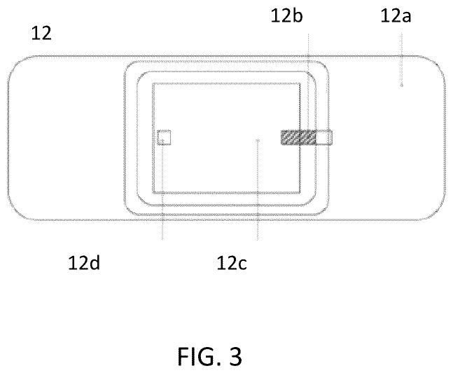

In embodiments, a nerve stimulator 12 may be connected to the needle 10, and specifically to the conducting surfaces 10b. In embodiments, the nerve stimulator 12 may be connected to the needle 10 to allow each one of the conductive surfaces or pads 10b to be driven individually. In embodiments, the stimulator 12 may also connect to the users' skin via an electrode or an array of transcutaneous electrodes 12a (see FIG. 3). In embodiments, the nerve stimulator 12 may provide an electrical signal to the conducting surfaces 10b.

In embodiments, each of the conducting surfaces 10b may be activated individually. In embodiments, the polarity of the current directed to each conducting surface 10b may be alternated (switched from positive to negative) and may be incrementally adjusted for each individual conducting surface 10b to provide for shaping of complex electric fields. Shaping of the field may be used to provide directional fields that are used to provide an estimate of the distance and direction of the needle tip 10a to the target nerve.

One or more feedback sensors 14 (see FIG. 1) may be provided to detect a response of the user's body to the directional fields generated by the conducting surfaces 10b driven by the stimulator 12. In embodiments, the feedback sensors 14 may be electrodes and may use electromyography (EMG) and/or acceleromyography (AMG) to provide stimulation information associated with the muscle activity in the muscle or muscles associated with the target nerve. For example, as generally shown in FIG. 1, the feedback sensors 14 may be positioned on or near the muscles that are affected by the target nerve that the needle 10 is inserted to affect.

As indicated in FIG. 1, the system 100 may be positioned on a user for use. In embodiments, the needle 10 may be inserted into the user's skin in the area of the target nerve. In embodiments, the nerve stimulator 12 may be secured to the user's skin, via adhesive electrode 12a and electrically connected to the needle 10, including the conducting surfaces 10b. In embodiments, a port 12b may be provided to provide a wired connection to the needle 10, or another external element that may be driven by electrical signals provided by the nerve stimulator 12. In embodiments, the feedback sensors 14 may be positioned over or in the vicinity of the muscles associated with the target nerve. In embodiments, a central controller 20 may be connected to the nerve stimulator 12 to control the electrical signals provided to the conducting surfaces 10b, and thus, to control the directional field generated by the needle. In embodiments, the central controller 20 may also be connected to the feedback sensors 14 to receive the stimulation information associated with the muscles associated with the target nerve. In embodiments, the connection between the central controller 20 and the stimulator 12 and/or the feedback sensors 14 may be wired or wireless. In embodiments the controller 20 may be integrated into the stimulator 12. In embodiments, the controller 10 may be integrated into the needle 10 along with the stimulator 12. In embodiments, the controller 20 may be or may be integrated into a portable electronic device such as a smart phone, tablet or laptop computer to name a few.

FIG. 3 illustrates a more detailed view of the stimulator 12 that may be used in the system 100 of FIG. 1. In embodiments, the stimulator 12 may include the adhesive electrode 12a in contact with the user's skin. In embodiments additional adhesive electrodes may be used. In embodiments, the connector or port 12b may be provided to provide an electric connection to the needle 10 or other device. In embodiments, a light emitting diode (LED) 12d may be included in or on the stimulator 12. In embodiments, the LED 12d may be provided to indicate operation of, or a mode of operation of, the stimulator. In embodiments, a translucent top 12c may be provided such that the LED 12 is visible through the top of the stimulator 12. In embodiments, a high voltage, preferably a maximum of 400V may be provided to the conducting surfaces 10b via the stimulator 12. In embodiments, the amplitude of the waveform provided to the conducting surfaces 10b via the stimulator may be between 0.1 and 5.0 milliamperes (mA), although higher maximum amperage may be used. In embodiments, the stimulator 12 may be similar to the Stimpod NMS 460, however, other nerve stimulating devices may be used.

In embodiments, the intensity and direction of directional electrical fields emanating from the needle 10, and specifically the conducting surfaces 10b, will elicit evoked responses in the form of fasciculations or even pre-fasciculation electrical activity in the muscles associated with the target nerve, which is detected and represented by the stimulation information provided by the feedback sensors 14, which may be or include an AMG/EMG system. The combination of the stimulation parameters, that is the characteristics of the signals sent to the conducting surfaces 10b and the stimulation information provided by the feedback sensors 14 may be used to provide an estimate of the distance and direction of the needle tip 10a from the target nerve. That is, based on the field created based on the stimulation parameters and the reaction of the patient's body to the field, the location of the needle tip relative to the target nerve may be determined.

In embodiments, the stimulation parameters and the stimulation information may be provided to the central controller 20, or a separate controller hub. In embodiments, the central controller 20 may be a processor, microprocessor or other control circuit or element operably connected to a memory. In embodiments, the central controller 20 may include or be operably connected to a display device 22. In embodiments, the controller 20 may be integrated into a housing of the display device 22 or otherwise connected thereto. In embodiments, the display device 22 may be integrated into or provided on a cart 24 to allow for mobility. In embodiments, the cart 24 may include a carousel 26 on which the display 22 may be provided. In embodiments, the cart 24 may include a hook or other attachment element 27 for holding headphones or other accessories and may include one or more storage bays or shelves 28 for holding other equipment, including additional stimulators 12 and/or feedback sensors 14. In embodiments, the central controller 20 may be or may include a mobile electronic device, such as a smart phone, tablet or laptop computer, to name a few. In embodiments, the central controller 20 may be or may be operably connected to a server or other remote computer system.

In embodiments, the central controller 20 processes the stimulation parameters and the stimulation information using algorithms which may include advanced signal processing techniques and/or artificial intelligence to determine distance information and direction information associated with the distance and direction that the needle tip 10a is distanced from the target nerve in three dimensions. In embodiments, the memory of the controller 20 may include processor executable code that when executed by the processor or microprocessor implements the signal processing or artificial intelligence algorithms to determine the distance information and direction information. In embodiments, as noted above, the simulation parameters may be provided to the stimulator 12 to provide a directional electromagnetic field emanating from the conducting sections 10b of the needles 10. Based on the direction of the field and position of the needle 10a, different reactions will be elicited in the muscle associated with the target nerve. The different reactions elicited in the muscle may be reflected by the stimulation information provided by the feedback sensors 14. In embodiments, the central controller 20 may also provide tissue information associated with the type of tissue that surrounds the needle tip 10a.

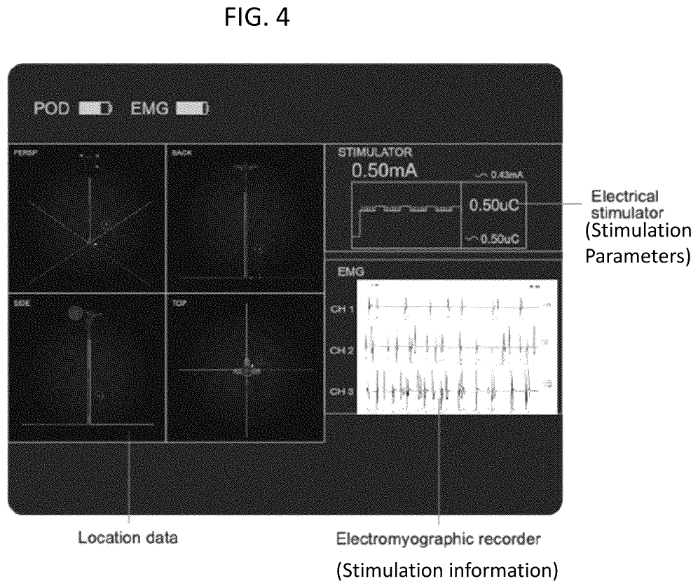

In embodiments, the distance information and direction information may be presented in visual form on the display 22 associated with the central controller 20. In embodiments, the distance information and direction information may be or may be used to generate image information that may be provided to the display 22 to present one or more images to the user showing the position of the needle (location information) relative to the target nerve in three dimensions. FIG. 4 illustrates an exemplary image that may be provided on the display 22 associated with the central controller 20 to show the position of the needle 10. As can be seen in FIG. 4 the image(s) may include or may be displayed with the stimulation data as well as the stimulation parameters. The display 22 may be a liquid crystal display (“LCD”), but may configured as a CRT, LED, plasma or other suitable display. In embodiments, operational controls may be graphically displayed on the display 22. In embodiments, the display 22 may be a touch-screen, and selections of various controls and stimulation parameters may be made by the user simply tapping or otherwise touching display. In embodiments, the central controller 20 may include or be connected to other input elements that may be used to enter information, such as a keyboard, mouse, to name a few.

In embodiments, the system 100 may be used to identify a target nerve and to provide RA using pharmaceuticals, which may be delivered to the target nerve via the needle 10, for example. As noted above, the pharmaceuticals used in regional anesthesia are difficult on the body due to their inherent toxicity. Further, there are a variety of shortcomings associated with general anesthesia as well. Thus, it would be advantageous to provide for anesthesia that avoids the shortcoming of general anesthesia and local anesthetic drugs.

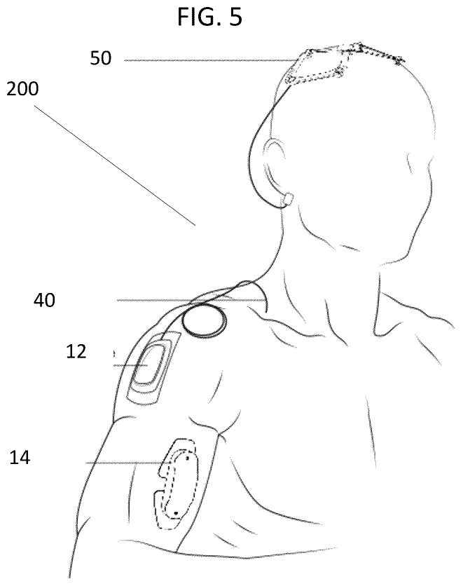

FIG. 1A illustrates an exemplary block diagram of the system 100 of FIG. 1 in which the stimulator 12 is connected to the needle 10 to provide electrical signals to the conducting surfaces 10b to provide the directions field. The controller 22 is connected to the stimulator 12 either by wire or wirelessly and the controller 22 is connected to the feedback sensors 14 either by wire or wirelessly. As noted above, the stimulation parameters may be provided to the stimulator 12 by the controller 22. The catheter 40 and EEG system 50 shown in broken lines in FIG. 1A are additional features that may be used to provide a system 200 (see FIG. 5) used to provide RA without utilizing pharmaceuticals and using electro anesthesia.

In embodiments, a system 200 may be used to provide RA without utilizing pharmaceuticals. In embodiments, a conductive catheter 40 (see FIG. 2C-2D, for example) may be fed through the needle 10 such that it extends beyond the distal end 10a of the needle 10 as can be seen in FIG. 2D, for example. In embodiments, an exposed conductive tip 40a of the catheter 40 may be provided at the end thereof extending beyond the end of the needle 10a and may be used to provide desired electrical signals to the target nerve to provide electro-anesthesia. In embodiments, the system 100, 200 may be used to provide visualization of the needle 10 for use in either pharmaceutical based regional anesthesia as discussed above or non-pharmaceutical electro-anesthesia using the catheter 40.

In embodiments, the present disclosure provides a system 200 and method that utilizes a proprietary waveform for delivering electrical percutaneous regional anesthesia which may be used with the visual guidance system discussed above. In embodiments, the proprietary waveform may include a low frequency square wave with a high frequency bi-polar exponentially decaying waveform superimposed thereon. In at least one embodiment, the high frequency component provides electrical signals with a frequency of about 133 kHz. In embodiments, other pain relieving waveforms may be used. In embodiments, the pain relieving waveform may include a low frequency component and a high frequency component. In embodiments, the electro anesthesia system 200 (see FIG. 5, for example) may also utilize evoked potentials such as electroencephalographic (EEG) information and/or brain stem response information as feedback information for confirming the success of anesthesia. In embodiments, other feedback information, such as compound action potentials, nociceptive reflex information, EMG information (such as that provided via the sensors 14), pupilometry information, and the like, may be used. In embodiments, the proprietary waveform may be delivered at a pulse repetition rate with a frequency in the range of 1 Hz to 10 kHz. In embodiments, the waveform may be provided in conjunction with the nerve stimulator 12 and the needle 10 and the signal is transmitted internally in the patient's body to provide regional anesthesia. Accordingly, a system and method are provided for delivering regional electro anesthesia as a function of a percutaneous application of a stimulating proprietary waveform.

In accordance with the present application, in embodiments, the proprietary wave signal includes a radio frequency component between 50 kHz and 500 kHz that provides percutaneous electrical stimulation for the delivery of regional anesthesia. In embodiments the proprietary wave signal may include a radio frequency component of 133 kHz to provide percutaneous electrical stimulation. The application of a radio frequency component to suppress pain transmission and muscular functions is a significant departure from known electro anesthesia/analgesia techniques. Radio frequency signals capitalize on different propagation structures within the nervous system than known Hodgkin Huxley models propose. This added element may influence the propagation of pain signals as well as their suppression in ways that have not been explored in known electro anesthesia/analgesia techniques.

Where electro anesthesia is used to provide regional anesthesia, the position of the needle 10 relative to the target nerve should be precise. In embodiments, a stimulating catheter. such as the catheter 40 discussed above. may be connected to a stimulator, such as the nerve stimulator 12 discussed above. This catheter 40 may be placed using technology such as the visual guidance system 100, but can also be placed using ultrasound and/or traditional nerve stimulating locating techniques.

For electro-anesthesia placement, the impedance/path of least resistance between the catheter tip 40a and the nerve is a key consideration which may or may not be directly reflected by proximity.

In embodiments, the system 200 may be used to provide regional electro anesthesia during surgery. In embodiments, the stimulator 12 may be connected to the catheter 40 (without the feedback sensor 14 (which may include an EMG/AMG sensor or EEG sensor). The catheter 40 may be driven by the stimulator 12 to provide a multitude of specific waveforms to induce paresthesia/anesthesia to the target nerve supplying the peripheral area to be subjected to surgery. Such waveforms may be delivered to include sub-threshold high frequency or low frequency pulse repetition rates as well as above threshold high frequency or low frequency pulse repetition rates. The threshold referred to here is associated with a perception threshold of the patient. That is, the threshold is associated with the patient's threshold for perceiving pain. In embodiments, the pulse repetition rates and amplitudes may vary based on the patient. In embodiments, the stimulator 12 may be controlled by the central controller 20 in much the same manner as discussed above with respect to system 100. In embodiments, the stimulator 12 may be controlled by a handheld smart device or other mobile electronic device which may be or may include the controller 20. In embodiments, the stimulator 12 may be controlled via a wireless connection, such as a smart phone, tablet or laptop to provide for remote control of the controller 20 to control the stimulator 12 to provide pain relief. In embodiments, the central controller 20 may be or may be included in the smart phone, tablet or laptop.

In embodiments, the stimulator 12 and the catheter 40 may be used along with the feedback sensors 14 (EMG/AMG sensors) as well as the EEG system 50, or other feedback to monitor the efficacy and placement of the catheter 40 during the surgical procedure by monitoring the evoked potential based on the motor nerve stimulation of the nerve bundle being anesthetized. As noted above, in embodiments, the feedback information may be based on compound action potentials.

In embodiments, the feedback sensor 14 which may provide EMG/AMG sensing, and may include an EMG sensor (with two electrodes touching the skin to measure the EMG signals) as well as an AMG sensor as noted above. In embodiments, the feedback sensor 14 may include signal conditioning/processing hardware, an analogue to digital converter, a processor, Bluetooth transceiver, status indicating LED's, and a rechargeable battery. In embodiments, the signal conditioning/processing hardware, analogue to digital converter and processor may be provided remotely, for example, in the controller 20 and may receive the EMG/AMG information from the electrode 14 and process it. In embodiments, the processed information may be used as the stimulation information discussed above and in the system 200.

In embodiments, the combination of AMG and EMG are useful as they measure different signals and compensate for their respective shortcomings. For example, AMG is prone to movement artifacts and struggles to pick up very small myographic signals, while EMG is subject to artifacts relating to electrostatic discharge as well as electromagnetic interference but immune to movement artifacts and is able to pick up very small myographic signals.

In embodiments, signal processing may be provided with respect to the stimulation information provided by the EMG and AMG as noted above. In embodiments, the signal processing may include:
1: Hardware noise reduction utilizing common mode rejection principles;
2: Amplification of the filtered signal;
3: Analog to digital conversion of the amplified signal.
4: Application of a non-linear noise reduction algorithm.
5: Combining the filtered EMG and AMG signals to provide the stimulation information which may be used to provide an accurate characterization of muscle activation due to the evoked potential introduced through the catheter 40 by the controller 20.

In embodiments, the combining step may include: a linear regression, a logistic regression a fuzzy logic classifier, a neural network, an Adaptive Neuro Fuzzy Inference System, a quadratic equation or any combination thereof.

In embodiments, the feedback sensors 14 (EMG/AMG) may provide feedback in the form of the stimulation information to the controller 20 indicative of the intensity of muscle activation due to the evoked potential triggered by the stimulator 12 and catheter 40. In embodiments, the stimulator 12, or controller 20, may adjust the simulating parameters (intensity and/or pulse width) in a closed loop fashion to ensure optimal engagement of the target nerve. That is, in embodiments, the stimulation information may be used to adjust the stimulation parameters to modify the waveform provided via the catheter 40 to the target nerve.

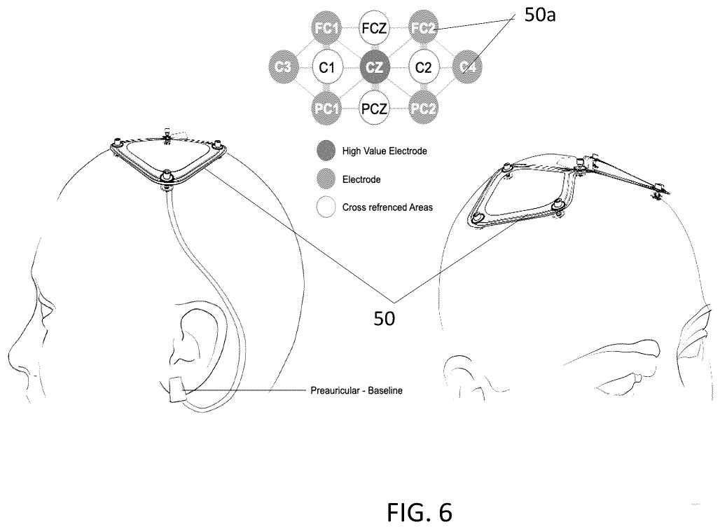

In embodiments, EEG feedback information may be used in the system 200. In embodiments, an EEG system 50 may monitor the nociceptive pain centers in the brain of the patient. In embodiments, the EEG system 50 may include an EEG sensor array 50a which includes a plurality of electrodes position around the user's head. In embodiments, the positioning of the electrodes may be indicated by the Modified Combinatorial Nomenclature (MCN) designations shown in FIG. 6.

In embodiments, the EEG system 50 may include or be connected to signal conditioning/processing hardware, an analogue to digital (A/D) converter, a processor, a Bluetooth transceiver, status indicating LED's and a rechargeable battery. In embodiments, the signal conditioning/processing hardware, A/D converter and processor may be provided separate from the sensor array 50a. for example in the controller 20. In embodiments, the signal conditioning may include:
1: reducing hardware noise using common mode rejection principles.
2: amplifying the filtered signal
3: providing the amplified signal to the analogue to digital converter.
4: processing the digitized signal information using a non-linear noise reduction algorithm using a localized processing unit.
5: combining filtered EMG and AMG signals and providing an accurate characterization of muscle activation due to the evoked potential introduced through the catheter by means of the stimulating unit.
   In embodiments, the step of combining may include: using linear regression, logistic regression, a fuzzy logic classifier, a neural network, an Adaptive Neuro Fuzzy Inference System, a quadratic equation or any combination thereof.

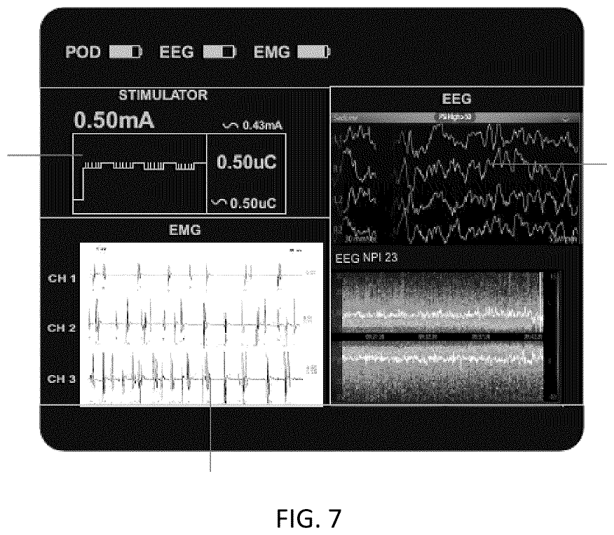

In embodiments, where the EEG system 50 is used, prior to surgery, a baseline nociceptive pain index (bNOC) may be established. In embodiments, the nociceptive pain index (NOC) may follow a simple count of 1-50 with 50 associated with the highest pain level and 1 the lowest. In embodiments, the NOC index information may be transmitted via Bluetooth to the controller 20, for example. In embodiments, the processed EEG information may be displayed, on the display 22, for example, as can be seen in FIG. 7, for example. In embodiment, the EEG information may be transmitted wirelessly to the central controller 20.

In embodiments, the central controller 20 may adjust the stimulating parameters provided to the stimulating unit 12 based on the pain index information. In embodiments, the adjusted stimulating parameters may be transmitted to the stimulator 12 via Bluetooth or any other suitable wireless communication system or protocol.

In embodiments, the stimulating parameters may include current intensity (generally in the range of 0.01 mA-5.0 mA), pulse width (generally in the range of 0.05 ms to 1.0 ms) and pulse repetition frequency (generally in the range of 1 Hz-10 kHz). In embodiments, the adjustment to the stimulating parameters may include decreasing or increasing one or more of these.

In embodiments, changes in the nociceptive pain index (NOC) from the baseline bNOC may be quantified by the central controller 20 and incremental adjustments to the stimulating parameters may be made repetitively until such time that the nociceptive pain index (NOC) information, based on the EEG information, indicates sufficient pain control, that is, a low pain level.

In embodiments, the system 200 may be used after surgery to provide pain relief as well. In embodiments, typically, after surgery, the feedback sensors 14 (AMG/EMG) and the EEG system 50 may be removed from the user's body. In embodiments, the stimulator 12 may be controlled based on input provided by the patient or another, via a remote control device, which may be a smart phone, tablet, laptop computer or any other electronic user device. In embodiments, the central controller 20 may be or may be integrated into the smart phone, tablet, laptop computer or other electronic user device as noted above. In embodiments, the remote control device may provide control signals, or stimulation parameters, to the stimulator 12 via wireless communication. In embodiments, the patient will be able to control the stimulator 12 with the remote handheld device (including the controller 20) to adjust stimulation to address the pain control requirements during the early post operative phase.

In embodiments, the feedback sensors 14 (AMG/EMG) and EEG system 50 or other feedback system may be configured to be worn by the user after surgery as well. In embodiments, the feedback sensors 14 (AMG/EMG) and EEG system 50 may be wearable and disposable, providing 24/7 real-time feedback to the handheld smart device and/or central controller 20. In embodiments, this information may be processed to provide automatic closed loop pain control as discussed above. In embodiments, this embodiment may also be used which will negate the need for user intervention. In embodiments, the feedback information from the feedback sensors 14 (AMG/EMG) and EEG system 50 may be used in conjunction with user input to provide pain management. In embodiments, the feedback information from the feedback sensors 14 (AMG/EMG) and EEG system 50 may be monitored by the surgeon remotely and used to monitor recovery of the patient including to help identify complications.

In embodiments, the stimulator 12 may be disconnected from the catheter 40 and switched out for another unit in the case of a malfunction and/or when the battery is depleted.

In embodiments, the stimulator 12 may be used to control the catheter 40 to stimulate the target nerve to re-train the neural connection and/or to strengthen the associated muscle during rehabilitation. In embodiments, control of the stimulator 12 may be automated and controlled by the central controller 20. In embodiments, the feedback sensor 14 (AMG/EMG) may be placed on the affected muscle in this instance to measure muscle deficiencies. In embodiments, the stimulation information provided by the feedback sensor 14 (AMG/EMG), may be used to measure nerve conduction velocity. In embodiments, the EEG system 50 may be used to provide EEG information that may be used to objectively measure the nociceptive pain component during the rehab process. In embodiments, a rehabilitation stimulation protocol may be administered by a physician or other health care professional providing control information using the handheld controller that the patient keeps with him/her. In embodiments, the rehabilitation stimulation protocol may vary depending on the surgery or procedure that has been performed and from which the patient is rehabilitating.

In embodiments, after the health care professional completes the session, the patient may be sent home with the stimulator 12 still attached but without the feedback sensors (EMG/AMG) and EEG system 50 with the handheld controller configured to allow the patient to apply the rehabilitation protocol while the patient is at home.

In embodiments, the feedback sensor 14 (AMG/EMG) and EEG system 50 may be wearable and may be used to provide 24 hour, 7 day a week feedback to the hand held controller or other controller 20. Such information can be sent back to the physician or therapist who will then be able to adjust rehabilitation protocols remotely. In embodiments, this information may be stored by the central controller 20 and/or may be periodically transmitted to a device associated with the physician or therapist who may use the information to track the patient's rehabilitation.

Although the present invention is described and shown in relation to particular embodiments thereof, many other variations and modifications and other uses will become apparent to those skilled in the art. Thus, various embodiments and variations are shown and described herein, and it is preferred, therefore, that the present invention be limited not by the specific disclosure herein.

What is claimed is:

1. A system for positioning a needle comprises:
a hollow needle configured to pierce a user's skin and including at least a first conductive surface adjacent to a point of the needle;
a stimulator electrically connected to the first conductive surface and configured to provide an electrical signal to the first conductive surface;
a feedback sensor configured to be secured to the user's skin and to provide stimulation information associated with activity of a muscle associated with a target nerve;
a controller operably connected to the stimulator and the feedback sensor and configured to:
provide stimulation parameters to the stimulator to control the electrical signal provided to the first conductive surface to provide a directional electrical field around the needle,
receive stimulation information from the feedback sensor; and
generate image information associated with a position of the needle in the user's body based on the stimulation parameters and stimulation information.

2. The system of claim 1, further comprising a display element operably connected to the controller and operable to receive the image information and provide an image of the needle in the user's body.

3. The system of claim 1, wherein the controller comprises:
a processor; and
memory operably connected to the processor and including processor executable code, that when executed by the processor:
generates the stimulation parameters based at least on the stimulation information; and
generates the image information based at least on the stimulation parameters and the stimulation information.

4. The system of claim 3, wherein the image information includes distance information and direction information associated with the needle and the target nerve.

5. The system of claim 4, where in the processor executable code, when executed by the processor, applies signal processing techniques to the stimulation information to generate the distance information and the direction information.

6. The system of claim 4, wherein the processor executable code, when executed by the processor, implements a machine learning algorithm to generate the distance information and the direction information based on the stimulation information and the stimulation parameters.

7. The system of claim 2, wherein the stimulation parameters and stimulation information are provided to the display element and illustrated on the display element.

8. The system of claim 2, wherein the display element is one of a liquid crystal display, cathode ray tube display, light emitting diode display and a plasma display.

9. The system of claim 2, wherein the display element is a touch screen configured to enter information associated with the stimulation parameters.

10. The system of claim 1, further comprising an input element configured to receive the stimulation parameters.

11. The system of claim 1, wherein the hollow needle is configured to provide a pharmaceutical anesthesia to the target nerve.

12. The system of claim 1, further comprising a catheter mounted in the needle with a distal end of the catheter extending beyond the hollow needle, the distal end including a conductive portion electrically connected to the stimulator, wherein the stimulator provides a pain relieving waveform to the conductive portion of the catheter for application to the target nerve.

13. The system of claim 12, wherein the pain relieving waveform includes a low frequency square wave with a high frequency bi-polar exponentially decaying waveform superimposed thereon.

14. The system of claim 12, wherein the high frequency hi-polar exponentially decaying waveform has a frequency of about 133 kHz.

15. The system of claim 12, wherein the pain relieving waveform includes a radio frequency component between 50 kHz and 500 kHz of varying or non-varying amplitude.

16. The system of claim 12, wherein the stimulation information indicates effectiveness of the pain relieving waveform based on activity of the muscle associated with the target nerve.

17. The system of claim 12, further comprising a plurality of EEG electrodes configured for attachment to a user's head and configured to provide EEG information indicating brain activity associated with pain of the user.

18. The system of claim 17, wherein the stimulation parameters are set based on the EEG information.

19. The system of claim 12, wherein the controller is operably connected to a user device associated with the user, wherein the user device provides control information to the controller to control the stimulator to provide the pain relieving waveform.

20. The system of claim 19, wherein the user device is connected to the controller via a wired connection.

21. The system of claim 19, wherein the user device is connected to the controller via a wireless connection.

22. The system of claim 17, wherein at least one of the EEG information, brain stem response information, compound action potentials, nociceptive reflex information, EMG information and pupilometry information is provided to the display element and illustrated on the display element.
